SEQUENCE LISTING (16 sequences)

SEQ ID NO: 1 - protein, 3 aa, Artificial
  Synthetic Construct
IGY

SEQ ID NO: 2 - protein, 3 aa, Artificial
  Synthetic Construct
IAY

SEQ ID NO: 3 - protein, 4 aa, Artificial
  Synthetic Construct
IPVE

SEQ ID NO: 4 - protein, 5 aa, Artificial
  Synthetic Construct
IEGTL

SEQ ID NO: 5 - protein, 4 aa, Artificial
  Synthetic Construct
IVDI

SEQ ID NO: 6 - protein, 9 aa, Artificial
  Synthetic Construct
VAPEEHPTL

SEQ ID NO: 7 - protein, 3 aa, Artificial
  Synthetic Construct
IVY

SEQ ID NO: 8 - protein, 3 aa, Artificial
  Synthetic Construct
  MISC_FEATURE (2)..(2): X is R, N, D, Q, E or K
IXY

SEQ ID NO: 9 - protein, 3 aa, Artificial
  Synthetic Construct
  MISC_FEATURE (3)..(3): X is Y, F or W
IAX

SEQ ID NO: 10 - protein, 3 aa, Artificial
  Synthetic Construct
  MISC_FEATURE (3)..(3): X is Y, F or W
IGX

SEQ ID NO: 11 - protein, 3 aa, Artificial
  Synthetic Construct
LGY

SEQ ID NO: 12 - protein, 3 aa, Artificial
  Synthetic Construct
LAY

SEQ ID NO: 13 - protein, 3 aa, Artificial
  Synthetic Construct
LVY

SEQ ID NO: 14 - protein, 3 aa, Artificial
  Synthetic Construct
  MISC_FEATURE (2)..(2): X is R, N, D, Q, E or K
LXY

SEQ ID NO: 15 - protein, 3 aa, Artificial
  Synthetic Construct
  MISC_FEATURE (3)..(3): X is Y, F or W
LAX

SEQ ID NO: 16 - protein, 3 aa, Artificial
  Synthetic Construct
  MISC_FEATURE (3)..(3): X is Y, F or W
LGX

(19) United States
(12) Patent Application Publication
(10) Pub. No.: US 2024/0300999 A1
(43) Pub. Date: Sep. 12, 2024

(54) PEPTIDES FOR REGULATING GLUCOSE

(71) Applicants: Thomas A. Gill, Elmsdale (CA); Jonathan Rolin, Hammonds Plains (CA)

(72) Inventors: Thomas A. Gill, Elmsdale (CA); Jonathan Rolin, Hammonds Plains (CA)

(21) Appl. No.: 18/573,089

(22) PCT Filed: Jun. 29, 2022
(86) PCT No.: PCT/CA2022/051035

§ 371 Date: Dec. 21, 2023

Related U.S. Application Data
(60) Provisional application No. 63/216,080, filed on Jun. 29, 2021.

(51) Int. Cl. C07K 5/083; A61K 38/00; A61P 3/10; C07K 1/16; C07K 1/34; C07K 14/46

(52) U.S. Cl. CPC C07K 5/0808; A61P 3/10; C07K 1/16; C07K 1/34; C07K 14/461; A61K 38/00

(57) ABSTRACT

Novel peptides and their uses are provided. In particular, the peptides are useful for modulating glucose uptake. The peptides are also useful for regulating glucose levels and/or treating diabetes in a subject.

### CROSS-REFERENCE TO RELATED APPLICATION

This disclosure claims the benefit and priority of U.S. Appl. No. 63/216,080, filed Jun. 29, 2021, the contents of which are incorporated herein by reference in their entirety.

### FIELD

This disclosure relates to novel glucoregulatory peptides and their use for modulating glucose uptake. The disclosure also relates to use of the peptides for treating diabetes.

### BACKGROUND

Type 2 diabetes (T2D) is a complex multifactorial disorder resulting from insulin resistance in peripheral tissues such as skeletal muscle, and pancreatic β-cell dysfunction (Stumvol et al., 2005). According to a recent report from the International Diabetes Federation, in 2000, 151 million people aged between 18 to 99 years had T2D. In 2017, 425 million people were suffering from T2D (International Diabetes Federation, 2017). This disease is growing at a fast rate (Wild et al., 2004).

Salmon Protein Hydrolysate (SPH) has been tested in in vitro studies. SPHs may have effects on glucose uptake (Chevrier et al., 2015, Roblet et al., 2016) and hepatic glucose production (Chevrier et al., 2015). These bioactivities may be caused by the presence of low molecular (<1 kDa) bioactive peptides (BPs) in the SPHs which have yet to be identified.

### SUMMARY

In this context, the inventors aimed to generate bioactive fractions useful for the treatment of Type 2 diabetes (T2D) and to identify potential peptide sequences responsible for this bioactivity.

Provided herein are glucoregulatory peptides, compositions, fractions, and combinations, and methods and uses thereof.

Accordingly, an aspect of the present disclosure includes a peptide comprising:
(i) an amino acid sequence selected from:
   (a) an amino acid sequence as shown in SEQ ID NO: 1 (IGY) or SEQ ID NO: 11 (LGY),
   (b) an amino acid sequence as shown in SEQ ID NO: 7 (IVY) or SEQ ID NO: 13 (LVY),
   (c) an amino acid sequence as shown in SEQ ID NO: 8 (IX1Y) or SEQ ID NO: 14 (LX1Y), where X¹ is a hydrophilic amino acid, optionally R, N, D, Q, E or K,
   (d) an amino acid sequence as shown in SEQ ID NO: 9 (IAX2) or SEQ ID NO: 15 (LAX2), where X² is F or W, and
   (e) an amino acid sequence as shown in SEQ ID NO: 10 (IGX3) or SEQ ID NO: 16 (LGX3), where X³ is Y, F or W; or
(ii) a peptide comprising at least 67% sequence identity with the amino acid sequence of any one of (a) to (e) that modulates glucose uptake.

Another aspect of the present disclosure includes a peptide comprising (i) an amino acid sequence as shown in SEQ ID NO: 1 (IGY) or SEQ ID NO: 11 (LGY); or (ii) a peptide comprising at least 67% sequence identity with the amino acid sequence as shown in SEQ ID NO: 1 or SEQ ID NO: 11 that modulates glucose uptake.

In one embodiment, the peptide comprises or consists of the amino acid sequence shown in SEQ ID NO: 1.

In another embodiment, the peptide comprises or consists of the amino acid sequence shown in SEQ ID NO: 11.

Another aspect of the present disclosure includes a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

In one embodiment, the peptide comprises or consists of the amino acid sequence shown in SEQ ID NO: 2.

In another embodiment, the peptide comprises or consists of the amino acid sequence shown in SEQ ID NO: 12.

In another embodiment, the peptide is less than 35, 30, 25, 20, 15, 10, 8, 6, 5 or 4 amino acids in length.

In another embodiment, the peptide is 3-10 amino acids in length. In a further embodiment, the peptide is 3, 4, 5 or 6 amino acids in length.

In another embodiment, the peptide is modified for cell permeability, stability or bioavailability.

Also provided is a composition comprising one or at least one of the peptides or fractions described herein and optionally a carrier.

Further provided is a composition or combination comprising a peptide as described herein and at least one additional peptide selected from:
(a) a peptide comprising (i) an amino acid sequence as shown in SEQ ID NO: 3 (IPVE); or (ii) a peptide comprising at least 75% sequence identity with the amino acid sequence as shown in SEQ ID NO: 3 that increases glucose uptake, and
(b) a peptide comprising (i) an amino acid sequence as shown in any one of SEQ ID NO: 4 (IEGTL), SEQ ID NO: 5 (IVDI) or SEQ ID NO: 6 (VAPEEHPTL); or (ii) a peptide comprising at least 67, 75, 80, or 90% sequence identity with the amino acid sequence as shown in any one of SEQ ID NOs: 4-6 that decreases hepatic glucose production, and optionally a carrier.

Further provided is an isolated fraction of salmon protein hydrolysate (SPH), wherein the fraction is obtained by fractionating salmon protein hydrolysate (SPH) by gel filtration chromatographic separation,
wherein the chromatographic separation is performed isocratically with 50 mM ammonium formate, pH 6.0 at a 0.1 mL/min flow rate, and
wherein the fraction modulates glucose uptake.

In one embodiment, the SPH is obtained by:
providing homogenized salmon;
precipitating protein from the homogenized salmon;
hydrolyzing the precipitated proteins to form a hydrolyzed solution;
filtering the hydrolyzed solution using an ultrafiltration membrane to generate the SPH.

In another embodiment, the fraction has glucose uptake stimulating activity at 1 μg/mL and 1 ng/ml in cultured L6 myotubes.

In another embodiment, the fraction comprises at least one peptide as described herein. In another embodiment, the fraction comprises at least one peptide comprising or consisting of amino acid sequence shown in SEQ ID NO: 1 (IGY) or comprising or consisting of amino acid sequence shown in SEQ ID NO: 2 (IAY). Further provided is a composition, optionally a nutraceutical composition, comprising at least one fraction as described here and optionally a carrier.

Yet a further aspect includes a method of increasing glucose uptake in a subject in need thereof, the method comprising administering to the subject a peptide, fraction, composition, or combination described herein.

A further aspect includes a method of regulating glucose levels in a subject in need thereof, the method comprising administering to the subject a peptide, fraction, composition, or combination described herein.

A further aspect includes a method of treating diabetes, optionally type 1 or type 2 diabetes, in a subject in need thereof, the method comprising administering to the subject a peptide, fraction, composition, or combination described herein.

A further aspect includes a method of treating metabolic syndrome (MS) by reducing hyperglycemia in a subject in need thereof, the method comprising administering to the subject a peptide, fraction, composition, or combination described herein.

A further aspect includes a method of providing antioxidant treatment to a subject in need thereof, the method comprising administering to the subject a peptide, fraction, composition, or combination described herein.

Yet a further aspect includes a method of lowering blood pressure and/or treating or preventing hypertension in a subject in need thereof, the method comprising administering to the subject a peptide, fraction, composition, or combination described herein.

In an embodiment, the subject is a diabetic subject.

In an embodiment, the subject is a mammal, optionally a dog, cat, horse, or human. In one embodiment, the subject is a human.

In an embodiment, the peptide, fraction, composition, or combination is administered or is for use orally, nasally or intravenously.

Also provided is a method of obtaining the peptides described herein, the method comprising:
providing a homogenized salmon frame or fraction;
precipitating proteins from the homogenized fraction;
hydrolyzing the precipitated proteins to form a hydrolyzed solution;
filtering the hydrolyzed solution using an ultrafiltration membrane to generate a filtrate; and
isolating the peptides from the filtrate.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### DRAWINGS

Embodiments are described below in relation to the drawings in which:

FIG. 1 shows GF Separation 4 of SPF on Bio-Gel P-2 media; 10 mm×300 mm column; flow rate, 0.1 mL per minute; eluant, 50 mM ammonium formate pH 6.0; detection at (A) 214 nm, (B) 254 nm and (C) 280 nm; sample concentration, 40 mg/ml; injection volume, 100 μL. Lines represent the UV absorbance at the corresponding wavelength. Fractions were collected within the indicated boundaries and pooled from n=8 separations.

FIG. 2 shows sequence logos of tripeptides in the glucose uptake inhibiting fraction 6 of GF Separation 4. Logos represent the frequency that indicated residues will be found located at that position. Logos were generated with WebLogo 3, using peptide sequences identified by software-assisted database searching and the modified validation criteria. Amino acid sequences are reported using single letter amino acid codes.

FIG. 3 shows (A) the extracted ion chromatogram at m/z 408.1-408.3 from fraction 6 of GF Separation 4 targeting the lle-lle-Tyr peptide ion and (B) the superimposed MS/MS spectra of each indicated precursor ion in panel A that resemble this peptide sequence. EIC were generated using Arcadiate software Version 4.5, and MS/MS spectra were generated using mMass software Version 5.5.

FIG. 4 shows (A) the extracted ion chromatogram at m/z 366.1-366.3 from fraction 6 of GF Separation 4 targeting the lle-Ala-Tyr peptide ion and (B) the superimposed MS/MS spectra of each indicated precursor ion in panel A that resemble this peptide sequence. EIC were generated using Arcadiate software Version 4.5, and MS/MS spectra were generated using mMass software Version 5.5.

FIG. 5 shows (A) the extracted ion chromatogram at m/z 352.1-352.3 from fraction 6 of GF Separation 4 targeting the lle-Gly-Tyr peptide ion and (B) the superimposed MS/MS spectra of each indicated precursor ion in panel A that resemble this peptide sequence. EIC were generated using Arcadiate software Version 4.5, and MS/MS spectra were generated using mMass software Version 5.5.

### DESCRIPTION OF VARIOUS EMBODIMENTS

Unless otherwise indicated, the definitions and embodiments described in this and other sections are intended to be applicable to all embodiments and aspects of the present disclosure herein described for which they are suitable as would be understood by a person skilled in the art.

Unless otherwise defined, scientific and technical terms used in connection with the present disclosure shall have the meanings that are commonly understood by those of ordinary skill in the art.

Terms of degree such as “about”, “substantially”, and “approximately” as used herein mean a reasonable amount of deviation of the modified term such that the end result is not significantly changed. These terms of degree should be construed as including a deviation of at least +5% of the modified term if this deviation would not negate the meaning of the word it modifies. All ranges disclosed herein are inclusive of the endpoints, and the endpoints are independently combinable with each other.

A “therapeutically effective amount” is intended to mean that amount of a compound that is sufficient to treat, prevent or inhibit a disease or condition such as T2D and/or hyperglycemia. The amount of a given compound of the present disclosure that will correspond to such an amount will vary depending upon various factors, such as the given compound, the composition, the route of administration, the type of disease or disorder, the identity of the subject or host being treated, and the like, but can nevertheless be routinely determined by one skilled in the art. In one embodiment, a “therapeutically effective amount” is an amount sufficient to have a desired effect on a subject, such as modulating glucose.

### Compositions of Matter:

### Peptides, Nucleic Acids, Vectors and Recombinant Cells

The disclosure provides peptides that have effects, such as to modulate glucose uptake. The peptides described herein can modulate glucose uptake in vitro or in vivo.

Modulating glucose uptake includes both an increase in glucose uptake and a decrease in in glucose uptake. An agent which increases and/or decreases glucose uptake can be referred to as an agent which modulates glucose uptake. For example, the present inventors showed that the peptides described herein increased glucose uptake in L6 skeletal muscle cells when applied at 1 ng/mL and reduced glucose uptake when applied at 1 pg/mL.

Glucose uptake can typically occur in one of two ways: passively (such as by facilitated diffusion) or actively (such as by secondary active transport).

An increase in glucose uptake by a cell refers to the increase in the amount, whether active or passive, of glucose that is taken up by the cell. A decrease in glucose uptake by a cell refers to the decrease in the amount, whether active or passive, of glucose that is taken up by the cell. Decreasing glucose uptake of a cell includes the reduction of uptake of glucose by the cell from the extracellular environment, e.g., from blood vessels or surrounding environment. Decreasing glucose uptake includes a reduction or decrease in the uptake of glucose by at least some cells of a subject.

The terms increase or higher refer to any increase above normal homeostatic levels. For example, control levels are in vitro, ex vivo, or in vivo levels prior to, or in the absence of, addition of an agent. Thus, the increase can be at least: 1.1, 1.2, 1.3, 1.4, 1.5, 1.6, 1.7, 1.8, 1.9 or 2.0 fold, or any amount of increase in between as compared to native or control levels.

The terms decrease or reduce refer to any decrease below normal homeostatic levels. For example, control levels are in vitro, ex vivo, or in vivo levels prior to, or in the absence of, addition of an agent. Thus, the decrease can be at least: 0.9, 0.8, 0.7, 0.6, 0.5, 0.4, 0.3, 0.2 or 0.1 fold, or any amount of decrease in between as compared to native or control levels.

Peptides provided by the present disclosure are set out in Tables 4 and 5 and include SEQ ID NOs: 1, 2 and 7-16.

As used herein, the term “peptide” refers to two or more amino acids linked by a peptide bond, and includes synthetic and natural peptides as well as peptides that are modified. Various lengths of peptides are contemplated herein.

The peptide can for example be 4-35 amino acids in length as amino acids may be added to the peptides in Table 4 and 5, optionally 4-10 amino acids in length or 4, 5, 6, 7, 8, 9 or 10 amino acids in length. The peptide can for example be any number of amino acids between 4 and 35.

In one embodiment, the peptide comprises or consists of:
(i) an amino acid sequence selected from:
   (a) an amino acid sequence as shown in SEQ ID NO: 1 (IGY) or SEQ ID NO: 11 (LGY),
   (b) an amino acid sequence as shown in SEQ ID NO: 2 (IAY) or SEQ ID NO: 12 (LAY),
   (c) an amino acid sequence as shown in SEQ ID NO: 7 (IVY) or SEQ ID NO: 13 (LVY),
   (d) an amino acid sequence as shown in SEQ ID NO: 8 (IX1Y) or SEQ ID NO: 14 (LX1Y), where X¹ is a hydrophilic amino acid, optionally R, N, D, Q, E or K,
   (e) an amino acid sequence as shown in SEQ ID NO: 9 (IAX2) or SEQ ID NO: 15 (LAX2), where X² is an aromatic amino acid, optionally F or W, and
   (f) an amino acid sequence as shown in SEQ ID NO: 10 (IGX3) or SEQ ID NO: 16 (LGX3), where X³ is an aromatic amino acid, optionally Y, F or W; or
(ii) a peptide comprising at least 67% sequence identity with the amino acid sequence of any one of (a) to (f) that modulates glucose uptake.

In particular, described herein is the peptide “IGY” comprising the amino acid sequence set out in SEQ ID NO: 1, or a conservatively substituted variant thereof, wherein the peptide modulates glucose uptake. For example, as isoleucine and leucine are very similar amino acids with identical masses, it is expected that peptide “LGY” (SEQ ID NO: 11) would have the same activity as “IGY”.

Also described herein is the peptide “IAY” comprising the amino acid sequence set out in SEQ ID NO: 2, or a conservatively substituted variant thereof, wherein the peptide modulates glucose uptake. For example, as isoleucine and leucine are very similar amino acids with identical masses, it is expected that peptide “LAY” (SEQ ID NO: 12) would have the same activity as “IAY”.

In another embodiment, the peptide comprises an amino acid sequence as shown in SEQ ID NO: 1 or SEQ ID NO: 2, or a conservatively substituted variant thereof.

Also provided is a peptide that is a part of a sequence described herein, optionally a part of SEQ ID NO: 1 or SEQ ID NO: 2, that retains all or part of the biological activity of a peptide having the amino acid sequence of SEQ ID NO: 1 or SEQ ID NO: 2. In one embodiment, the biological activity is modulation of glucose uptake.

In another embodiment, the peptide consists essentially of, or consists of an amino acid sequence as shown in SEQ ID NO: 1 or SEQ ID NO: 2, or a conservatively substituted variant thereof.

In another embodiment, the peptide comprises an amino acid sequence with at least 67% sequence identity with the amino acid sequence as shown in any one of SEQ ID NO: 1, 11, 2 or 12.

The peptide comprising SEQ ID NO: 1, 11, 2 or 12 may further comprise additional amino acids and be at least: 4, 5, 6, 7, 8, 9, 10, 11, 12, 13, 14, 15, 16, 17, 18, 19, or 20 amino acids in length. In an embodiment, the peptide is less than 50, 45, 40, 35, 30, 25, 20, 15, 14, 13, 12, 11, 10, 9, 8, 7, 6 or 5 amino acids in length and comprises an amino acid sequence encoding a peptide that modulates glucose uptake as described herein, such as SEQ ID NO: 1, 11, 2 or 12.

In one embodiment, the disclosure provides a peptide that has at least: 67% sequence identity with SEQ ID NO: 1, 11, 2 or 12.

Sequence identity can be calculated according to methods known in the art. Sequence identity is optionally assessed by the algorithm of BLAST version 2.1 advanced search. BLAST is a series of programs that are available, for example, online from the National Institutes of Health. The advanced blast search is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_141; Altschul, S. F., Madden, T. L., Schiffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656. In addition, percent identity between two sequences may be determined by comparing a position in the first sequence with a corresponding position in the second sequence. When the compared positions are occupied by the same nucleotide or amino acid, as the case may be, the two sequences are conserved at that position. The degree of conservation between two sequences is often expressed, as it is here, as a percentage representing the ratio of the number of matching positions in the two sequences to the total number of positions compared.

As used herein, the term “conservatively substituted variant” refers to a variant with at least one conservative amino acid substitution. A “conservative amino acid substitution” as used herein, refers to the substitution of an amino acid with similar hydrophobicity, polarity, and R-chain length for one another. In a conservative amino acid substitution, one amino acid residue is replaced with another amino acid residue without abolishing the protein's desired properties. Without the intention of being limited thereby, in one embodiment, the substitutions of amino acids are made that preserve the structure responsible for the ability of the peptide to increase glucose uptake or decrease hepatic glucose production as disclosed herein. Examples of conservative amino acid substitutions include:

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

In one embodiment, the peptides described herein are optionally modified for cell permeability, improved stability, and/or better bioavailability. These modifications include, without limitation, peptide conjugation, peptide cyclization, peptide end modification (e.g. N-acetylation or C-amidation, side chain modifications including the incorporation of non-coded amino acids or non-natural amino acids, N-amide nitrogen alkylation, chirality changes (incorporation of or replacement of L-amino acids with D-amino acids), generation of pseudopeptides (e.g. amide bond surrogates), or peptoids, or azapeptides or azatides). In one embodiment, the peptides described herein are modified by the addition of a lipophilic moiety.

The peptides described above may be prepared using recombinant DNA methods. These peptides may be purified and/or isolated to various degrees using techniques known in the art. Accordingly, nucleic acid molecules having a sequence which encodes a peptide of the disclosure may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule encoding a peptide of the disclosure and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The peptides may be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

In one embodiment, the peptides may be modified with a detectable label. For example, in one embodiment the peptide is fluorescently, radioactively or immunologically labeled.

The peptides may also be modified with an enhancer moiety. Accordingly, another aspect provides a compound comprising a peptide described herein and an enhancer moiety. In one embodiment, the peptide is conjugated directly or indirectly to the enhancer moiety. As used herein, an enhancer moiety can increase or enhance the activity of the peptide. For example, the enhancer may be a permeability enhancer, a stability enhancer or a bioavailability enhancer. The enhancer moiety is optionally selected from a protein carrier, or a polymer carrier. In one embodiment, the enhancer moiety is a carrier protein, thereby forming a fusion protein. In another embodiment, the enhancer moiety is a PEG moiety.

The peptides may also be modified with a cell-penetrating moiety. As used herein, the term “cell-penetrating moiety” refers to a moiety that promotes cellular uptake of the peptide upon delivery to a target cell. Examples of cell-penetrating moieties include cell-penetrating peptides that translocate across the plasma membrane of eukaryotic cells at higher levels than passive diffusion. In one embodiment, the cell-penetrating peptide can translocate the nuclear membrane of a cell to enter the nucleus. In another embodiment, the cell-penetrating peptide can enter the nucleolus.

In one embodiment, the cell-penetrating peptide is an amphipathic peptide comprising both a hydrophilic (polar) domain and a hydrophobic (non-polar) domain. Cell-penetrating peptides can include sequences from membrane-interacting proteins such as signal peptides, transmembrane domains and antimicrobial peptides.

The peptides described herein can also be conjugated to a carrier protein, thereby forming a fusion protein.

The disclosure also includes nucleic acids that encode the peptides described herein. As used herein, the term “nucleic acids” includes isolated nucleic acids. In one embodiment, the disclosure provides nucleic acids that encode a peptide comprising or consisting of SEQ ID NO: 1 or SEQ ID NO: 2 or any peptide described herein.

In another embodiment the disclosure provides a nucleic acid having at least 50, 60, 67, 70, 80, 90, 95 or 99% sequence identity with a nucleic acid that encodes a peptide comprising or consisting of SEQ ID NO: 1 or SEQ ID NO: 2 or any peptide described herein, a nucleic acid that hybridizes to a nucleic acid that encodes a peptide comprising or consisting of SEQ ID NO: 1 or SEQ ID NO: 2 or any peptide described herein under at least moderately stringent hybridization or stringent hybridization conditions.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C. . . . 16.6 (Log 10 (Na⁺)+0.41(% (G+C)-600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in Ausubel, 1989 and in Sambrook et al., 1989.

The disclosure further contemplates a vector comprising a nucleic acid described herein, optionally a recombinant expression vector containing a nucleic acid molecule that encodes a peptide of the disclosure and the necessary regulatory sequences for the transcription and translation of the inserted protein-sequence. In an embodiment, the vector is a viral vector such as a retroviral, lentiviral, adenoviral or adeno-associated viral vector.

Recombinant expression vectors can be introduced into host cells to produce a transformed host cell for the purpose of producing the peptides described herein. The term “transformed host cell” is intended to include prokaryotic and eukaryotic cells which have been transformed or transfected with a recombinant expression vector of the disclosure. The terms “transformed with”, “transfected with”, “transformation” and “transfection” are intended to encompass introduction of nucleic acid (e.g. a vector) into a cell by one of many possible techniques known in the art. Suitable host cells include a wide variety of prokaryotic and eukaryotic host cells.

Also provided in another aspect is a recombinant cell expressing a peptide, nucleic acid, vector or compound described herein. In an embodiment, the cell is a bacterial cell, yeast cell, a mammalian cell, or a plant cell.

### Bioactive Fractions

The disclosure also provides bioactive fractions, or extracts, obtained from salmon that have effects, such as to modulate glucose uptake. The fractions described herein can modulate glucose uptake in vitro or in vivo.

In one embodiment, the disclosure provides an isolated fraction of salmon protein hydrolysate (SPH), wherein the fraction is obtained by fractionating salmon protein hydrolysate (SPH) by gel filtration chromatographic separation, wherein the chromatographic separation is performed isocratically with 50 mM ammonium formate, pH 6.0 at a 0.1 mL/min flow rate. Optionally, the fraction is a bioactive fraction that modulates glucose uptake.

As used herein, the term “salmon protein hydrolysate (SPH)” refers to salmon that is minced and hydrolyzed. Hydrolyzation may be performed by treating the salmon with trypsin, chymotrypsin, pepsin, or any combination thereof.

Salmon protein hydrolysate (SPH) may be obtained by any method known in the art including the methods described herein and described in Jin (2012).

In one embodiment, the SPH is obtained by:
providing homogenized salmon;
precipitating protein from the homogenized salmon;
hydrolyzing the precipitated proteins to form a hydrolyzed solution; and
filtering the hydrolyzed solution using an ultrafiltration membrane to generate the SPH.

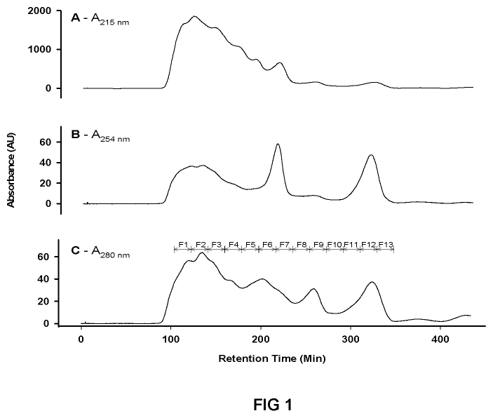

In another embodiment, the separation is performed on Bio-Gel P-2 media with a 10 mm×300 mm column and a flow rate of 0.1 mL per minute with a sample concentration of 40 mg/ml and injection volume of 100 UL and the fraction corresponds to:
fraction 2 of FIG. 1 (retention time of approximately 20-40 min),
fraction 3 of FIG. 1 (retention time of approximately 40-60 min),
fraction 6 of FIG. 1 (retention time of approximately 100-120 min),
fraction 7 of FIG. 1 (retention time of approximately 120-140 min),
fraction 8 of FIG. 1 (retention time of approximately 140-160 min), or
fraction 9 of FIG. 1 (retention time of approximately 160-180 min).

In another embodiment, wherein the fraction has glucose uptake stimulating activity. Glucose uptake stimulating ability may be measured as known in the art and as described herein. For example, in one embodiment, the fraction has glucose uptake stimulating activity at 1 μg/mL and 1 ng/mL as assayed in cultured L6 myotubes.

In another embodiment, the fraction comprises at least one peptide as described herein. In a further embodiment, the fraction comprises at least one peptide comprising or consisting of amino acid sequence shown in SEQ ID NO: 1 (IGY) or comprising or consisting of amino acid sequence shown in SEQ ID NO: 2 (IAY).

The fraction is optionally lyophilized, for example by freezing and lyophilizing the fraction.

Also provided is a combination of two or more fractions as described herein.

### Compositions and Combinations of Peptides

The disclosure also provides a composition comprising one or more of the peptides described herein. Also provided is a combination of two or more peptides described herein. Further provided is a composition comprising one or more of the fractions described herein.

In one aspect, the composition comprises a peptide described herein and a carrier. In another embodiment, the composition or combination comprises a peptide comprising or consisting of SEQ ID NO: 1 or SEQ ID NO: 11 and a peptide comprising or consisting of SEQ ID NO: 2 or SEQ ID NO: 12 and optionally a carrier.

In another aspect, the composition comprises a peptide described herein and at least one additional peptide that also modulates glucose uptake and optionally a carrier. In one embodiment, the composition comprises a peptide described herein and
(a) a peptide comprising (i) an amino acid sequence as shown in SEQ ID NO: 3 (IPVE); or (ii) a peptide comprising at least 75% sequence identity with the amino acid sequence as shown in SEQ ID NO: 3 that increases glucose uptake, or
(b) a peptide comprising (i) an amino acid sequence as shown in any one of SEQ ID NO: 4 (IEGTL), SEQ ID NO: 5 (IVDI) or SEQ ID NO: 6 (VAPEEHPTL); or (ii) a peptide comprising at least 67, 75, 80, or 90% sequence identity with the amino acid sequence as shown in any one of SEQ ID NOs: 4-6 that decreases hepatic glucose production,
and optionally a carrier.

The disclosure also provides a composition comprising a peptide as described herein at a concentration or dose which modulates glucose uptake in a subject in need thereof, an optionally a carrier.

In one embodiment, the carrier is a carrier acceptable for administration to humans.

As used herein, the term “acceptable carrier” is intended to include any and all solvents, dispersion media, coatings, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions and dextrose solution.

In one embodiment, the carrier is a nanoparticle. The nanoparticle may optionally allow for oral and/or nasal administration in an aerosol, vapor, mist or spray.

In one embodiment, a composition or combination described herein is formulated to be compatible with its intended route of administration. Examples of routes of administration include oral, nasal and parenteral, e.g. intravenous, intradermal, subcutaneous.

For example, in one embodiment, the active ingredient such as a peptide described herein is prepared with a carrier that will protect it against rapid elimination from the body, such as a sustained/controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, see for example Li et al, 2015.

In one embodiment, oral, nasal or parenteral compositions or combinations are formulated in dosage unit form for ease of administration and uniformity of dosage. Dosage unit form as used herein refers to physically discrete units suited as unitary dosages for the subject to be treated; each unit containing a predetermined quantity of active ingredient calculated to produce the desired therapeutic effect in association with the required carrier. The specification for the dosage unit forms are dictated by and directly dependent on the unique characteristics of the active ingredient and the particular therapeutic effect to be achieved, and the limitations inherent in the art of preparing such an active ingredient for the treatment of individuals.

In one embodiment, the compositions described herein comprise an agent that enhances its function, such as, for example, insulin, other diabetes medication(s), omega 3, and/or polyphenols. The composition can also contain other active ingredients as necessary or beneficial for the particular indication being treated, optionally those with complementary activities that do not adversely affect each other. Such active ingredients are suitably present in combination in amounts that are effective for the purpose intended.

### Methods and Uses

The disclosure also provides uses and methods relating to the peptides, fractions, compositions, and combinations described herein.

The peptides disclosed herein modulate glucose uptake by cells, while others decrease hepatic glucose production. Accordingly, the peptides, fractions, compositions, and combinations of the present disclosure are useful for regulating blood glucose levels in a subject and optionally for treating diabetes in a subject. In one embodiment, the peptides described herein are useful for reducing hyperglycemia in a subject, optionally in a subject with T2D.

In one embodiment, the methods and uses include the administration to a subject or use in a subject of a peptide, fraction, composition or combination as described herein. In one embodiment, the subject is a diabetic subject. In one embodiment, the subject is a mammal, optionally a dog, cat, horse, or human. In one embodiment, the mammal is a human. In one embodiment, the peptide, fraction, composition, or combination is administered orally or intravenously. In another embodiment, the peptide, fraction, composition, or combination is for use orally, intravenously or nasally (for example, via an aerosol containing nanoparticles loaded with the peptide preparation).

Methods and uses of increasing glucose uptake:

The disclosure provides a method of increasing glucose uptake in a subject in need thereof, the method comprising administering to the subject a peptide, composition, fraction, or combination described herein. Also provided is use of a peptide, composition, or combination disclosed herein to increase glucose uptake. In another embodiment, a peptide, fraction, composition, or combination disclosed herein is used in the manufacture of a medicament to increase glucose uptake. In yet another embodiment, a peptide, fraction, composition, or combination disclosed herein is for use in treating hyperglycemia.

As used herein, the term “hyperglycemia” refers to higher than normal fasting blood glucose concentration, optionally at least 125 mg/dL.

### Methods and Uses of Regulating Glucose Levels:

The disclosure further provides a method of regulating glucose levels in a subject in need thereof, the method comprising administering to the subject a peptide, fraction, composition, or combination described herein. Also provided is use of a peptide, fraction, composition, or combination disclosed herein to regulate glucose levels. In another embodiment, a peptide, fraction, composition, or combination disclosed herein is used in the manufacture of a medicament to regulate glucose levels. In yet another embodiment, a peptide, fraction, composition, or combination disclosed herein is for use in regulating glucose levels.

Regulating glucose levels comprises the lowering of hyperglycemic glucose levels to a normoglycemic range. Optionally a normoglycemic range is 70-130 mg/dL. Optionally the glucose levels are maintained substantially in that normoglycemic, for example for at least: 30, 60, 90, 120, 180 or 240 minutes. For example, 30-60, 30-120, or 30-240 minutes.

### Methods and Uses of Treating Prediabetes:

The disclosure further provides a method of treating prediabetes in a subject in need thereof, the method comprising administering to the subject a peptide, fraction, composition, or combination described herein. Also provided is use of a peptide, fraction, composition, or combination disclosed herein to treat prediabetes. In another embodiment, a peptide, fraction, composition, or combination disclosed herein is used in the manufacture of a medicament to treat prediabetes. In yet another embodiment, a peptide, fraction, composition, or combination disclosed herein is for use in treating prediabetes.

Prediabetes is also referred to as “impaired glucose tolerance” or “impaired fasting glucose” and refers to blood glucose levels that are higher than a normal fasting blood glucose concentration, but are not high enough to be classified as type-2 diabetes. For example, from 100 to 125 mg/dL.

### Methods and Uses of Treating Diabetes:

The disclosure further provides a method of treating diabetes, optionally type 1 or type 2 diabetes, in a subject in need thereof, the method comprising administering to the subject a peptide, fraction, composition, or combination described herein. Also provided is use of a peptide, fraction, composition, or combination disclosed herein to treat diabetes, optionally type 1 or type 2 diabetes. In another embodiment, a peptide, fraction, composition, or combination disclosed herein is used in the manufacture of a medicament to treat diabetes, optionally type 1 or type 2 diabetes. In yet another embodiment, a peptide, fraction, composition, or combination disclosed herein is for use in treating diabetes, optionally type 1 or type 2 diabetes.

### Methods and Uses of Treating Metabolic Syndrome:

The disclosure provides a method of treating metabolic syndrome in a subject in need thereof by reducing one or more of hyperglycemia and hypertension, the method comprising administering to the subject a peptide, fraction, composition, or combination described herein. Also provided is use of a peptide, fraction, composition, or combination disclosed herein to treat metabolic syndrome. In another embodiment, a peptide, fraction, composition, or combination disclosed herein is used in the manufacture of a medicament to treat metabolic syndrome. In yet another embodiment, a peptide, fraction, composition, or combination disclosed herein is for use in treating metabolic syndrome.

### Methods and Uses of Providing Antioxidant Treatment:

It has been shown that salmon protein hydrolysate (SPH) and peptide fractions thereof have antioxidant activity (Girgih et al., 2013). Accordingly, the disclosure provides a method of providing antioxidant treatment and/or preventing or reducing damage from free radicals to a subject in need thereof, the method comprising administering to the subject a peptide, fraction, composition, or combination described herein. Also provided is use of a peptide, fraction, composition, or combination disclosed herein to provide antioxidant treatment and/or prevent or reduce damage from free radicals. In another embodiment, a peptide, fraction, composition, or combination disclosed herein is used in the manufacture of a medicament to provide antioxidant treatment and/or to prevent or reduce damage from free radicals. In yet another embodiment, a peptide, fraction, composition, or combination disclosed herein is for use in providing antioxidant treatment and/or preventing or reducing damage from free radicals.

### Methods and Uses of Treating Hypertension:

It has been shown that salmon protein hydrolysate (SPH) lowers blood pressure (Girgih et al., 2016). Accordingly, the disclosure provides a method of lowering blood pressure and/or treating or preventing hypertension in a subject in need thereof, the method comprising administering to the subject a peptide, fraction, composition, or combination described herein. Also provided is use of a peptide, composition, or combination disclosed herein to lower blood pressure and/or treat or prevent hypertension. In another embodiment, a peptide, fraction, composition, or combination disclosed herein is used in the manufacture of a medicament to lower blood pressure and/or treat or prevent hypertension. In yet another embodiment, a peptide, fraction, composition, or combination disclosed herein is for use in lowering blood pressure and/or treating or preventing hypertension.

### Methods of Obtaining Peptides:

The disclosure further provides a method of obtaining the peptides disclosed herein. In one embodiment the method comprises providing a homogenized salmon frame or fraction, precipitating proteins from the homogenized fraction, hydrolyzing the precipitated proteins to form a hydrolyzed solution, filtering the hydrolyzed solution using an ultrafiltration membrane to generate a filtrate, and isolating the peptides from the filtrate, optionally isolating peptides of SEQ ID NO: 1 and SEQ ID NO: 2 into separate fractions.

In an embodiment, precipitating the proteins is performed by isoelectric precipitation at pH 4.5.

Hydrolysis of precipitated proteins may be carried out with a variety of enzymes known to a person skilled in the art.

In an embodiment, hydrolyzing the peptides precipitated proteins is performed using trypsin, chymotrypsin, pepsin, or any combination thereof.

Ultrafiltration may comprise several techniques known to a skilled person. In an embodiment, ultrafiltration comprises-pressure driven ultrafiltration. In another embodiment ultrafiltration comprises electrodialysis with an ultrafiltration membrane.

Ultrafiltration membranes comprise pores that may be, for example, 0.1 to 0.001 μm.

In an embodiment, the ultrafiltration membrane has a nominal molecular weight cutoff of 1 kDa.

Peptide isolation may be performed using a variety of methods known to a skilled person and may include various chromatography methods such as size-exclusion, affinity purification, and ion exchange.

In an embodiment, isolating the peptides is performed using reverse-phase liquid chromatography.

Also provided is a method of producing a peptide as described herein comprising culturing a host cell that expresses a nucleic acid encoding the peptide, such as a peptide selected from SEQ ID NO: 1 and SEQ ID NO: 2, and optionally isolating the peptide.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Preparation of SPF (Salmon Peptide Fraction)

The SPF was prepared following protocol 2, as previously described in Jin (2012), with modifications. Briefly, mechanically deboned salmon mince was homogenized with 1.0 M NaOH at a 1:4 (w/v) ratio in a blender on high speed for 2 min, then stirred for 2 h. The pH was then adjusted to 4.5 using 2.0 M HCl to isoelectrically precipitate the protein. The isoelectric precipitate was kept at 4° C. overnight to make sure the precipitation was complete. The precipitated proteins were pelleted by centrifugation at 5200×g for 20 min at 4° C. in a Sorvall RC-3 refrigerated centrifuge (Sorvall Instruments Div., Dupont Co., Newtown, CT, USA). The supernatant was discarded, and the pellet was resuspended in the same volume H₂O as NaOH during homogenization with vigorous shaking. The pH of this protein dispersion was adjusted to 2.0 using 2.0 M HCl in preparation for pepsin digestion. Pepsin (EC 3.4.23.1, Millipore Sigma Cat. No.: P6887, 3,200-4,500 units/mg protein) was added at an enzyme:substrate (E:S) ratio of 1:100, assuming the protein content in salmon muscle was 15% of the wet weight, and the average pepsin activity was 1000 units/mg protein. Enzyme-substrate mixtures were continuously stirred overnight at 37° C. The pH of the pepsin digest was then adjusted to 7.8 using 2.0 M NaOH to irreversibly deactivate the pepsin, and to prepare for trypsin and chymotrypsin digestion. Trypsin (EC 3.4.21.4) and Chymotrypsin (EC 3.4.21.1) mixture (Cat. No.: PHAM-378, 1:1 trypsin:chymotrypsin, 1000 units/mg protein, Creative Enzymes, Shirley NY, USA) were also added at an E:S ratio of 1:100 and stirred continuously for 4 h at 37° C. Reactions were terminated by heating to 100° C. for 10 min. The digests were centrifuged at 5200×g for 30 min at 4° C. and the supernatant was filtered using Whatman #1 filter paper through a Celite cake to remove any insoluble material. The filtrate was subsequently filtered using a Prep/Scale Tangential Flow Filtration (TFF) 2.5 ft² (0.232 m²) cartridge with a 1 kDa exclusion limit (Millipore Corporation, Bedford, MA, USA). The permeate fraction was collected, demineralized by electrodialysis, lyophilized, and stored at −30° C. until further use. Otherwise all steps for SPF production were performed as described by Jin (2012).

### Fractionation of the SPF Using Gel Filtration Chromatography

Gel filtration (GF) chromatographic separation of the SPF was performed using an ÄKTAexplorer 10 XT FPLC system and Unicorn V4.12 software, equipped with a Frac-950 fraction collector (GE Healthcare, Chicago, IL, USA) to fractionate potential bioactive peptides. A Tricorn column (GE Healthcare, Chicago, IL, USA; 10×300 mm) was packed in-house with Bio-Gel P-2 GF media (1800 Da-100 Da exclusion limit) (Bio-Rad Laboratories, Hercules, CA, USA). Freeze-dried SPF was suspended in 50 mM ammonium formate pH 6.0 to 40 mg/mL, filtered using 0.2 μm Whatman syringe filters, then 100 ul were loaded into the sample loop. Chromatography was performed isocratically with 50 mM ammonium formate, pH 6.0 at a 0.1 mL/min flow rate, until 1.75 column volumes had flowed through the column. UV absorbance was measured at 214, 254, and/or 280 nm wavelengths, and a total of 13 subfractions of 2 mL each were collected, Repeated fractionations of n=8 were performed. Eluted peptides from repeated fractionations were pooled, then freeze-dried, weighed and stored at −30° C. until in vitro screening.

### In Vitro Screening Assay for SPF Action on Metabolism

Screening assays were performed as follows. L6 rat myoblasts (courtesy of Dr. Amira Klip, Hospital for Sick Children, Toronto, Canada) were grown and maintained in monolayer culture in alpha-MEM containing 2% (v/v) fetal bovine serum in an atmosphere of 5% CO₂ at 37° C. L6 myoblasts were plated in 12-well or 24-well plates at 20,000 cells/mL, replacing the media every two days until complete differentiation into myotubes (7 days post-plating).

Once completely differentiated, myotubes were serum deprived (alpha-MEM with 0% FBS) for 3 h and treated or not with SPF (1 ng/ml or 1 μg/mL) for 2 h without (insulin-independent) or with 100 nM insulin (insulin-dependent) during the last 45 min. Cells were rinsed once with glucose-free HEPES-buffered saline solution pH 7.4 (140 mM NaCl, 20 mM HEPES/Na, 5 mM KCl, 2.5 mM MgSO₄ and 1 mM CaCl₂)), and were subsequently incubated for 8 min with 10 UM 2-deoxy-D-glucose containing 0.3 μCi/mL 2-deoxy-D-[3H]glucose in the same buffer. After incubation in transport medium, cells were rinsed three times with ice-cold saline solution (0.9% NaCl) and stored at −20° C. Cells were disrupted by adding 1 mL of 50 mM NaOH to plates with agitation for 15 min. Cell-incorporated radioactivity was measured using a Perkin Elmer Tricarb liquid scintillation counter. Protein concentrations were determined by the micro bicinchoninic acid method (BCA) using a bovine serum albumin (BSA) standard curve (ThermoScientific, Waltham, Mass, USA), and the results expressed in pM/min/mg protein, calculated using the following equation:

$$\frac{DPM ( sample )}{C \times DPM ( 2 DG ) \times t}$$

where DPM (sample) is the number of disintegrations per minute (DPM) measured for the tested sample, C is the concentration of protein (mg), DPM (2DG) is the number of DPM measure for the solution of radioactive 2-deoxy-D-[3H] glucose for 1 pmol and equal to 72.2025 dpm/pmol, and t is the incubation time with 2-deoxy-D-[3H] glucose, and reported in terms of relative activity to the control sample in the absence of insulin. Statistical analysis was performed in Microsoft Excel using a two-tailed Student's t-test assuming equal variance.

### Preparation of Salmon Protein Isoelectric Precipitates

Isoelectric precipitates of salmon protein were prepared from previously frozen, Atlantic salmon loin muscle, ground using a Moulinex household meat grinder and homogenized with 1.0 M, 0.5 M, 0.25 M or 0.1 M NaOH at a 1:4 (w/v) ratio in a standard blender on high for 30 s at high speed, then stirred for 2 h, or as indicated. Salmon muscle solutions were centrifuged at 3,500×g for 10 min at 4° C. using an IEC Centra MP 4R centrifuge (International Equipment Company, Chattanooga, TN, USA) and supernatants were transferred to a clean beaker. Proteins were precipitated by adjustment to pH 4.5 using 6.0 M HCl and were then centrifuged at 3,500×g for 10 min at 4° C. The pellet was lyophilized then kept at −30° C. until later use but the supernatant protein was precipitated by using ice cold acetone at a 1:4 (v/v) ratio and incubated under freezing conditions for 30 min. The precipitated proteins from supernatants were lyophilized at kept at −30° C. until later use.

### Sodium Dodecyl Sulfate Polyacrylamide Gel Electrophoresis

Sodium dodecyl sulfate polyacrylamide gel electrophoresis (SDS-PAGE) was performed using an LKB 2001 vertical electrophoresis unit coupled to an LKB 2297 Macro Drive 5 and an LKB 2219 Multitemp II Thermostatic Circulator (LKB-Produkter AB, Bromma, Sweden). Gels were poured in-house into assembled cassettes (16×16×0.15 cm) with a 38.4 mL volume. Three stock solutions were used to prepare the gels: solution A (30% acrylamide (w/v), 0.8% (w/v) bisacrylamide in dH₂O, 37.5:1), solution B (0.5 M Tris-HCl, pH 6.8), and solution C (3.0 M Tris-HCl, pH 8.8) (Bio Rad Laboratories, Hercules, CA), as well as a 10% SDS (Millipore Sigma, St. Louis, MO) and 1% ammonium persulfate (AMMO) solutions (Bio Rad Laboratories, Hercules, CA). Gels were prepared following a variation of the manufacturer's recipe to a volume of 30 mL for resolving gels, and 10 mL for stacking gels, per cassette. Gradient resolving gels (5-15%) were prepared using an LKB gradient gel former with light (2.5 mL Solution A, 2.0 mL Solution B, 10.0 mL dH₂O, 0.15 mL SDS, 0.35 mL AMMO, 0.04 mL TEMED) and heavy (7.5 mL Solution A, 2.0 mL Solution B, 5.0 mL dH₂O, 0.15 mL SDS, 0.35 mL AMMO, 0.02 mL TEMED) acrylamide solutions, while the stacking gel (1.25 mL Solution A, 2.5 mL Solution C, 6.25 mL dH₂O, 0.1 mL SDS, 0.5 mL AMMO, 0.04 mL TEMED) was poured isocratically. Alternatively, 15% resolving gels were prepared using 15.0 mL Solution A, 4.0 mL Solution B, 10.0 mL dH₂O, 0.15 mL SDS, 0.35 mL AMMO, and 0.04 mL TEMED. All solutions were degassed for five min before the addition of the SDS, AMMO and TEMED to prevent foaming and premature polymerization. The resolving gel was left to polymerize for 1 h, after which the stacking gel solution was poured with an inserted 10-well comb and left to polymerize overnight. Reservoir buffer was prepared from a 10× stock solution (144 g glycine, 30.3 g tris, 10 g SDS/L) to a final volume of 5.5 L.

Protein samples were prepared to 2 mg/mL and diluted 1.25-fold with sample buffer (0.1 M Tris-HCl, 10% β-mercaptoethanol (v/v), 8% SDS (w/v), 33% glycerol (v/v), 0.05% bromophenol blue (w/v)), heated to 70° C. for 15 min, then centrifuged at 5,250×g for 10 min at 4° C. using an IEC Centra MP 4R centrifuge (International Equipment Company, Chattanooga, TN, USA), and 30 μL were loaded into each well. Precision Plus Protein™ All Blue Prestained Protein Standards were used for the MW ladder (Cat No. 1610373, Bio Rad Laboratories, Hercules, CA) and 20 μL were loaded directly to the well. Each run was operated at a constant temperature of 15° C. and followed a constant-voltage electrophoretic treatment, starting at 100 V for 1 h or until the dye reached the stacking/resolving gel interface, and then increased to 300 V and terminated when the dye front reached 1 cm from the end of the gel. Following the completion of the run, the gel was transferred to a glass dish and washed three times with enough dH₂O to cover the gel surface, for 5 min each. Fixing solution (45:10:55, methanol:acetic acid:water) was added to cover the gel, and left overnight at 4° C. The gel was washed once with dH₂O and stained for 1 h with staining solution (10% (v/v) acetic acid, 0.025% (w/v) Coomassie Brilliant Blue R-250 (Bio Rad Laboratories, Heracles, CA, USA) with shaking for 1 h. The gel was destained with water and heating to 60° C. for 2 h, replacing the solution after one hour. Destaining continued until sufficient minimization of background was achieved. Gel images were recorded using a ChemiDoc XRS+ System and processed using the Image Lab Software (Bio Rad Laboratories, Hercules, CA).

### Protein Identification: LC-MS/MS Method 1

Excised gel slices stained with Coomassie Brilliant Blue R-250 were processed for in-gel digestion as previously described, with slight modifications (Shevchenko et al., 2007). Briefly, gel slices were washed for 2 h in dH₂O and then cut into ˜1 mm cubes and rinsed twice with 200 μL of dH₂O. Gel cubes were reduced with 10 mM dithiothreitol (DTT) at 56° C. for 30 min, then alkylated with 55 mM iodoacetamide for 30 min at room temperature in the dark, and then dehydrated with 200 μL acetonitrile (ACN). Dried gel cubes were saturated with 20 μg/mL of trypsin protease (Cat. No.: 90057, PierceTM ThermoFisher Scientific, Waltham, Mass, USA) for 2 h, then 20 μL of 50 mM ammonium bicarbonate was added and the samples were incubated overnight at 37° C. Digested peptides were extracted from the gel cubes by treatment with 100 μL of 50% ACN in 5% formic acid. The peptide-containing solution was dried to a pellet in a vacuum centrifuge and subsequently resuspended in 20 μL of a 3% ACN, 0.5% formic acid solution, and processed as outlined.

Gel bands were subject to analysis by LC-MS/MS at the Dalhousie University Biological Mass Spectrometry Core Facility on a VelosPRO orbitrap mass spectrometer (ThermoFisher Scientific, Waltham, Mass, USA) equipped with an UltiMate 3000 Nano-LC system (ThermoFisher Scientific, Waltham, Mass, USA). Chromatographic separation of the digests was performed on a PicoFRIT C18 self-packed 75 mm×60 cm capillary column (New Objective, Woburn, MA) at a flow rate of 300 nL/min. MS and MS/MS data was acquired using a data-dependent acquisition method in which a full scan was obtained at a resolution of 30,000, followed by ten consecutive MS/MS spectra in both higher-energy collisional dissociation (HCD) and collision-induced dissociation (CID) mode (normalized collision energy 36%). Internal calibration was performed using the ion signal of polysiloxane at m/z 445.120025 as a lock mass.

Raw MS data were analyzed using Proteome Discoverer Version 2.1 (ThermoFisher Scientific, Waltham, Mass, USA). The Sequest HT program was used to compare peak lists to the Salmonideae UniprotKB protein database as well as the CRAP database of common contaminants (Global Proteome Machine Organization), based on their tryptic cleavage for a minimum peptide length of 6, with tolerance for two missed cleavages. Cysteine carbamidomethylation was set as a fixed modification, while methionine (Met) oxidation, N-terminal Met loss, and phosphorylation on serine, threonine, and tyrosine were included as variable modifications. A mass accuracy tolerance of 10 ppm was used for precursor ions, while 0.02 Da for HCD fragmentation was used for product ions. The Percolator program was used to determine confident peptide identifications using a 0.1% false discovery rate (FDR). Parent proteins reporting Sequest HT scores >10 were accepted.

### Peptide Detection: LC-MS/MS Method 2

LC-MS/MS analyses were performed at Laval University using a 1290 Infinity II UPLC (Agilent Technologies, Santa Clara, CA, USA) consisting of a binary pump (G7120A), a multisampler (G7167B), an in-line degasser and a variable wavelength detector (G7114B) adjusted to 214 nm. The sample was loaded (10 μL) onto an Acquity UPLC CSH 130 1.7 μm C18 column (2.1×150 mm i.d., Waters Corporation, Milford, MA, USA). The column was operated at a flow rate of 400 ρL/min at 45° C. A linear gradient consisting of solvent A (LC-MS grade water with 0.1% formic acid) and solvent B (LC-MS grade ACN with 0.1% formic acid) was applied, with solvent B going from 2% to 25% in 50 min, holding until 53 min, ramping to 90% and holding until 57 min, then back to initial conditions.

A hybrid ion mobility quadrupole TOF mass spectrometer (6560 high definition mass spectrometry (IM-Q-TOF), Agilent, Santa Clara, USA) was used to identify and quantify the relative abundances of the peptides. Signals were recorded in positive mode at Extended Dynamic Range, 2 Ghz, 3200 m/z with a scan range between 100-3200 m/z. Nitrogen was used as the drying gas at 13.0 L/min and 150° C., and as nebulizer gas at 30 psig (0.207 MPa). The capillary voltage was set at 3500 V, the nozzle voltage at 300 V and the fragmentor at 400 V. The instrument was calibrated using an ESI-L low concentration tuning mix (G1969-85000, Agilent Technologies, Santa Clara, CA, USA). Data acquisition and analysis was performed using the Agilent MassHunter Software package (LC-MS/MS Data Acquisition, Version B.07.00 and Qualitative Analysis for IM-MS, Version B.07.00 with BioConfirm Software) to compare detected ions to the NCBI Salmo salar protein database, based on a no enzyme, pepsin, and/or trypsin:chymotrypsin cleavage with a minimum peptide length of three and tolerance for two missed cleavages. Variable modifications for oxidized methionine, pyroglutamic acid, deamination of Asp, and phosphorylation to Ser, Thr, and Tyr, were tolerated by the database searching algorithm. Precursor ions between 100-3,200 Da were selected for MS/MS. A mass accuracy tolerance of 20 ppm was used for precursor ions, while 50 ppm was used for product ions. The Agilent MassHunter Find by Molecular Feature algorithm was performed with Molecular Feature Extraction (MFE) as a pre-processing step to identify features (peptides), ions distinct from background noise, from within the raw MS spectral data. Compound lists for each bioactive fraction were calculated, reporting the mass, retention time, and relative intensity of significant precursor ions detected during LC-MS, but contained no sequence information. The validation of database searching was performed using modified validation criteria; peptides with scores >7, or % SPI >70, or minimum spectrum intensity of 1.0×10⁶, and simultaneously identified by the MFE algorithm, were considered valid peptide sequences.

### Sequence Logos

Sequence logos (Schneider and Stephens, 1990) were prepared using the WebLogo 3 web-application (http://weblogo.threeplusone.com/) (Crooks et al., 2004). Branched chain amino acids (lle, Leu, Val) were coloured blue, anionic residues (Asp, Glu) were coloured green, cationic residues (Arg, His, Lys) were coloured red, aromatic amino acids (Phe, Trp, Tyr) were coloured purple and all others black. The frequency was expressed as the fraction of the indicated residue at each position, and peptides were reported according to their length using single letter amino acid codes.

### Peptide Identification: In Silico Digestion of SPF Progenitor Proteins

The high-quality prediction of compositions for each bioactive fraction using the Agilent MassHunter Find by algorithm with Molecular Feature Extraction (MFE) was used to support the identification of peptide sequences. An in-house peptide database was developed resulting from in silico digestions of ten potential SPF progenitor proteins: myosin heavy chain, alpha actin, tropomyosin, creatine kinase, glyceraldehyde-3-phosphate dehydrogenase, fructose-bisphosphate aldolase A, triosephosphate isomerase, phosphoglycerate mutase, myosin light chain, and beta enolase, and selected due to their identification as progenitors to peptides identified in SAX Separation 1 and as components of the low-alkali salmon muscle precipitate. Each primary sequence was processed by specific or non-specific hydrolysis rules explained below to enable the annotation of each compound list generated for bioactive SPF fractions by the MFE with putative peptide identities, independent of software-assisted database searching.

Specific. The first in-house peptide database was generated using the Peptide Mass tool (SIB, Swiss Institute of Bioinformatics; Artimo et al., 2012), and represents peptide sequences generated through the activities of pepsin or trypsin and chymotrypsin, and allowing for up to 3 missed cleavages. The masses for each peptide sequence predicted by the in silico digestion of SPF progenitor proteins were directly compared to the masses calculated by the MFE algorithm for each bioactive fraction, where matching masses represented a putative identification corresponding to the sequence from the in-house database.

Non-Specific. A second in-house database was generated using the FindPep Tool (SIB, Swiss Institute of Bioinformatics; Artimo et al., 2012) and represents any peptide sequence from within SPF progenitor primary sequences that match the masses of MFE-calculated ions in bioactive fractions. In contrast to the ‘specific’ database, the FindPep Tool searches the complete primary sequence of progenitor proteins for matching peptide sequences and is not limited to only those peptides generated by the specificity of enzymatic activity. Theoretical sequences were matched to experimental precursor ions with a mass tolerance of 10 ppm.

### Peptide Identification: De Novo Sequencing

De novo sequencing was facilitated using Arcadiate software Version 4.5 and mMass software Version 5.5 (Strohalm et al., 2008). Arcadiate was used to generate base peak chromatograms of each bioactive fraction and to identify the precursor ions represented by dominant peaks. The MS/MS spectra of each precursor ion generated by fragmentation were visualized using mMass and its interpretation represented a putative identification. Sequences for each MS/MS spectra were informed by identifying masses unique to each of the 20 standard amino acids, with assistance from theoretical calculations performed by the mMass software. The MS/MS spectra of each putative peptide identification by this approach were exported to Sigma Plot Version 11 (San Jose, CA, USA) for additional annotation of the product ions.

### Validation of Potential Bioactive Peptides

Synthetic peptides lle-Ala-lle (4.2 mg; 99.59% purity), lle-Gly-lle (4.3 mg; 99.47% purity), lle-lle-lle (4.1 mg; 98.56% purity), lle-Ala-Tyr (4.3 mg; 98.48% purity), lle-Gly-Tyr (4.1 mg; 98.73% purity) and lle-lle-Tyr (4.2 mg; 99.19% purity) were purchased from Bio Basic Canada Inc. (Markham, ON, Canada) and were validated by the manufacturer by HPLC-MS/MS.

### Results and Discussion

Potential bioactive peptides in the SPF were investigated using gel filtration and strong anion exchange chromatography formats to identify mediators of glucose uptake in the SPF. Fractions generated from these separations were demonstrated to exhibit both stimulating and inhibiting modulation of glucose uptake in cultured L6 myotubes, and the absence of compositional similarities between functional fractions strongly suggested that SPF activity was mediated by distinct peptides. Glucose uptake stimulation was observed in fractions abundant in tripeptides and/or larger oligopeptides, containing both C- and N-terminal BCAAs, and/or anionic amino acids that were frequently positioned as consecutive pairs and often at the penultimate position (adjacent to the C-terminal amino acid). These characteristics could represent common motifs responsible for glucose uptake.

In contrast, glucose uptake inhibition was observed in SPF fractions containing di- and tripeptides with aromatic amino acids positioned at the C-terminus. Peptide identification by database searching was fraught with difficulties possibly associated with extensive false positive and false negative reporting. However, in silico digestions of the curated protein library combined with database searching was able to mitigate some of the challenges associated with the identification of short peptides. Comparing putative bioactive sequences from SPF fractions to previously reported bioactive peptides was unsuccessful, particularly considering that glucose uptake was mediated by SPF at concentrations as low as 1 ng/ml. Evaluating the peptide compositions of each fraction still enabled the identification of compositional features that could be used to uniquely describe aspects of functional peptide fractions.

In particular, the lle-X-lle motif was derived from fraction 3 of GF Separation 4 that exhibited stimulating activity on glucose uptake, while the lle-X-Tyr motif was derived from fraction 6 of GF Separation 4 that exhibited inhibiting activity on glucose uptake. A decision was made to procure chemically synthesized peptides following these potential bioactivity-mediating criteria to validate their individual glucose uptake modulating activities and the importance of each motif. In both fractions, peptide sequences representing these motifs were identified containing Ala, Gly, and lle located at the center amino acid position. The peptides lle-Ala-lle (m/z 316.223), lle-Gly-Ile (m/z 302.207), lle-lle-lle (m/z 358.270), lle-Ala-Tyr (m/z 366.202), lle-Gly-Tyr (m/z 352.187), and lle-lle-Tyr (m/z 408.249) were therefore selected for chemical synthesis and their glucose uptake modulating activities were evaluated.

### Peptide Selection

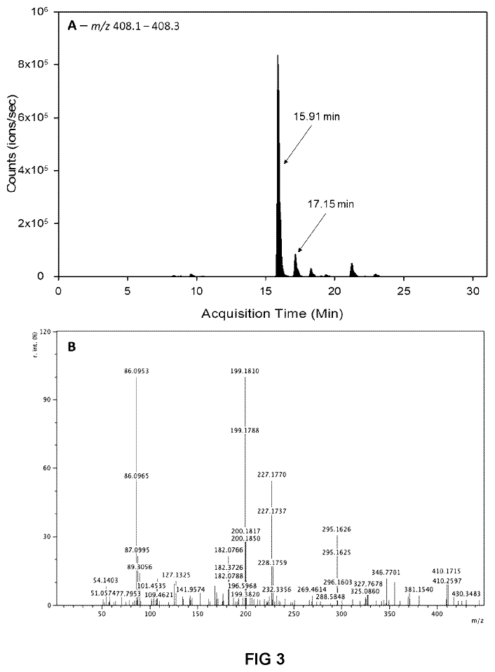

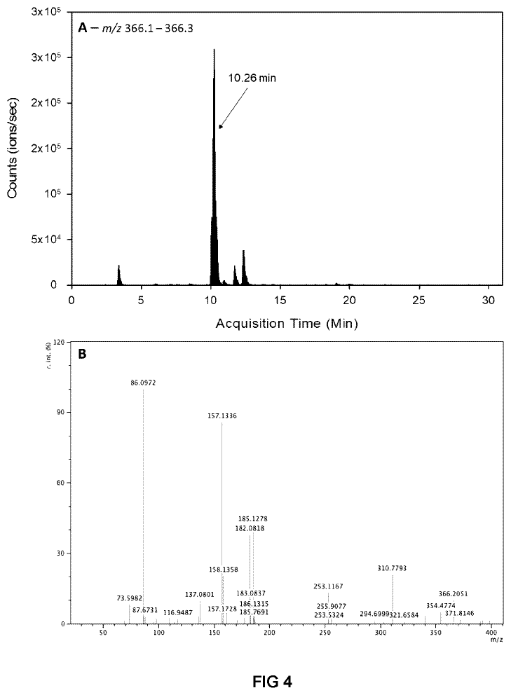

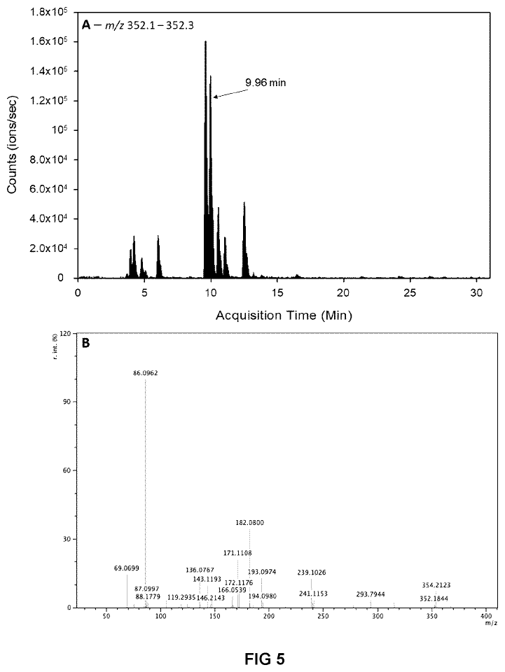

The putative bioactive peptide sequences selected for chemical synthesis were identified by software-assisted database searching and validated using the modified criteria. Peptides identified by this methodology have since been demonstrated to be of low quality (accuracy with respect to peptide identification) as a result of the challenges met by software to differentiate the sequences of precursor ions with identical masses but different RT's, and to accurately interpret the sequences of peptides thought to contain isobaric residues, such as lle and Leu, or that do not fragment into the typical b- and y-series ions. Therefore, to evaluate whether the selected peptides for chemical synthesis were in fact present in bioactive fractions of SPF, extracted ion chromatograms (EIC) (FIG. 3A-5A) that represent the MS detector response within a narrow m/z range and MS/MS spectra (FIG. 3B-5B) that represent the peptide fragmentation pattern, were generated corresponding to each putative bioactive peptide sequence.

### Glucose Uptake Analysis of Synthetic Peptides

The SPF from which these peptides were generated exhibited potent glucose uptake stimulating activity at 1 μg/mL and 1 ng/ml in cultured L6 myotubes (Table 2). Chemically synthesized peptides lle-Ala-lle, Ile-Gly-lle, and lle-lle-lle based on the lle-X-lle motif were evaluated at 1 μg/mL and/or 1 ng/ml. No significant effect on glucose uptake was observed for any of these synthetic peptides screened individually at 1 ng/ml, but when all peptides were combined at 1 μg/mL each (3 μg/mL total), inhibition of glucose uptake in the presence of insulin was observed (p-value=0.0473).

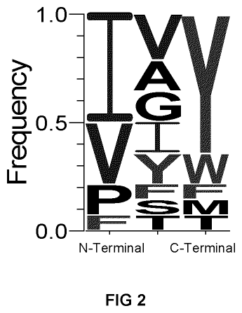

Chemically synthesized peptides lle-Ala-Tyr, lle-Gly-Tyr, and lle-lle-Tyr based on the lle-X-Tyr motif (FIG. 2) were tested (Table 3) and it was found that lle-Ala-Tyr peptide stimulated glucose uptake in the absence (p-value=0.0279) and presence (p-value=0.0256) of insulin at 1 ng/mL, whereas lle-Gly-Tyr only stimulated glucose uptake in the presence of insulin at 1 ng/ml (p-values=0.001).

lle-lle-Tyr did not significantly affect (p-value >0.05) glucose uptake activity in the presence or absence of insulin, when tested at 1 ng/ml. A mixture of the three tripeptides containing 1 ng/ml of each peptide (3 ng/ml total) inhibited glucose uptake (p-value=0.0170) in the absence of insulin, while testing at 1 μg/mL each (3 μg/mL total) had an inhibitory effect in the presence of insulin (p-value=0.0153).

At 1 ng/ml, the stimulation of glucose uptake was only observed for peptides that contained the C-terminal Tyr residue, therefore the activities of each of the three lle-X-Tyr peptides were further evaluated at 1 pg/mL. At this diluted concentration, lle-Ala-Tyr actually inhibited glucose uptake in the absence (p-value=0.0036) and presence (p-value=0.0070) of insulin, while lle-Gly-Tyr also inhibited glucose uptake but only in the presence of insulin (p-value=0.0435). A mixture of the three peptides (lle-Gly-Tyr, lle-Ala-Tyr and lle-lle-Tyr) containing 1 pg/mL each (3 pg/mL total) also maintained the inhibition of glucose uptake (p-value=0.0075) in the absence of insulin similarly observed at 3 ng/ml.

The lle-X-lle motif was identified from a peptide mixture with glucose uptake stimulating activity, but the activities of lle-Ala-lle, lle-Gly-lle and lle-lle-lle did not yield this effect.

In contrast, the lle-X-Tyr motif was identified from a peptide mixture with glucose uptake inhibiting activity, but purified peptides lle-Ala-Tyr and lle-Gly-Tyr had a stimulating effect when applied at 1 ng/ml and an inhibiting effect at 1 pg/mL. Regardless of concentration, lle-Ala-Tyr and lle-Gly-Tyr both maintained their activity targeting conditions where insulin is absent and present, respectively.

According to Song et al. (2017), the modulation of glucose uptake by bioactive peptides typically occurs as a result of their effect on AMPK activation in the absence of insulin or by their effect on Akt signaling pathways in response to insulin stimulation. Also, according to Song et al. (2017), these two effects can occur simultaneously. Without being bound by theory, this data may indicate that the bioactivities of lle-Ala-Tyr and lle-Gly-Tyr occurs through their effects on different cellular targets.

### SUMMARY

The present study shows that chemically synthesized peptides lle-Ala-Tyr and lle-Gly-Tyr, derived from bioactive fractions of the SPF, were validated as glucose uptake modulating peptides in cultured L6 myotubes. Ile-Ala-Tyr and lle-Gly-Tyr exhibited stimulation of glucose uptake in L6 myotubes at 2.7 and 2.8 nM, respectively, but inhibition at 2.7 and 2.8 pM. Without being bound by theory, novel mechanisms of glucose uptake modulation were suspected by each peptide due to their specificity to conditions without and with insulin-stimulation, and the modulating effects of mixtures containing these peptides could not be predicted by their activities when evaluated independently.

TABLE 1
Glucose uptake modulation of fractions from GF Separation
4 on Bio-Gel P-2 media. Fractions were each tested at
1 ng/mL in the (−) absence and (+) presence of insulin.

Glucose Uptake (fold activity to control)

| Sample | Insulin (−) | Insulin (+) |
|---|---|---|
| Control | 1.00 ± 0.00 | 1.99 ± 0.03 |
| Fraction 1 | 1.08 ± 0.08 | 2.18 ± 0.13 |
| Fraction 2 | 1.10 ± 0.05 | 2.38 ± 0.11* |
| Fraction 3 | 1.06 ± 0.04 | 2.24 ± 0.09* |
| Fraction 4 | 0.99 ± 0.05 | 2.11 ± 0.07 |
| Fraction 5 | 1.00 ± 0.05 | 2.16 ± 0.11 |
| Fraction 6 | 0.91 ± 0.04* | 1.77 ± 0.08* |
| Fraction 7 | 1.08 ± 0.03* | 2.19 ± 0.08* |
| Fraction 8 | 1.08 ± 0.02* | 2.11 ± 0.06 |
| Fraction 9 | 0.94 ± 0.04 | 1.71 ± 0.06* |
| Fraction 10 | 0.98 ± 0.06 | 2.08 ± 0.13 |
| Fraction 11 | 1.00 ± 0.08 | 2.13 ± 0.14 |
| Fraction 12 | 0.95 ± 0.04 | 1.89 ± 0.09 |
| Fraction 13 | 1.05 ± 0.06 | 2.15 ± 0.15 |

Values are means ± SEM, from n = 8 individual experiments, performed in triplicate;
*p-value < 0.05 vs. control.

TABLE 2
Glucose uptake modulation SPF processed following
Jin (2012) protocol 2. Fractions were each tested
in the (−) absence and (+) presence of insulin.

Glucose Uptake
(fold activity to control)

| Sample | Concentration | | Insulin (−) | Insulin (+) |
|---|---|---|---|---|
| Control | — | | 1.00 ± 0.00 | 1.80 ± 0.04 |
| SPF | 1 | ng/mL | 1.27 ± 0.04* | 2.20 ± 0.10* |
| SPF | 1 | μg/mL | 1.27 ± 0.03* | 2.23 ± 0.10* |

Values are means ± SEM, from n = 6-8 individual experiments, performed in triplicate;
*p-value < 0.05 vs. control.

TABLE 3
Glucose uptake modulation of chemically synthesized peptides representing
the Ile-X-Ile and Ile-X-Tyr motifs, at the indicated peptide concentration,
in the (−) absence or (+) presence of insulin.

| | Glucose Uptake (fold activity to control) | | | | | |
|---|---|---|---|---|---|---|
| | Concentration | | | | | |
| | 1 μg/mL | | 1 ng/mL | | 1 pg/mL | |
| Sample | Insulin (−) | Insulin (+) | Insulin (−) | Insulin (+) | Insulin (−) | Insulin (+) |
| Control | 1.00 ± 0.00 | 1.76 ± 0.03 | 1.00 ± 0.00 | 1.76 ± 0.03 | 1.00 ± 0.00 | 1.72 ± 0.02 |
| Ile-Ile-Ile | — | — | 1.00 ± 0.04 | 2.02 ± 0.27 | — | — |
| Ile-Ala-Ile | — | — | 1.04 ± 0.04 | 1.80 ± 0.09 | — | — |
| Ile-Gly-Ile | — | — | 1.04 ± 0.04 | 1.75 ± 0.17 | — | — |
| Combined | 0.91 ± 0.07 | 1.56 ± 0.07* | 1.08 ± 0.05 | 1.80 ± 0.15 | — | — |
| Ile-Ile-Tyr | — | — | 1.06 ± 0.03 | 1.88 ± 0.11 | 0.97 ± 0.04 | 1.61 ± 0.07 |
| Ile-Ala-Tyr | — | — | 1.13 ± 0.05* | 1.97 ± 0.08* | 0.86 ± 0.04* | 1.55 ± 0.04* |
| Ile-Gly-Tyr | — | — | 1.05 ± 0.06 | 2.02 ± 0.05* | 0.98 ± 0.06 | 1.49 ± 0.10* |
| Combined | 0.98 ± 0.07 | 1.48 ± 0.09 | 0.86 ± 0.05* | 1.70 ± 0.13 | 0.91 ± 0.03* | 1.59 ± 0.06 |

Values are means ± SEM, from n = 6 individual experiments, performed in triplicate.
*p-value < 0.05 vs. control.

TABLE 4
Identified peptides

| Peptide | | |
|---|---|---|
| Sequence | Bioactivity | SEQ ID NO: |
| IGY | Modulates glucose uptake | 1 |
| IAY | Modulates glucose uptake | 2 |

TABLE 5
List of Sequences

| SEQ | |
|---|---|
| ID NO: | Sequence |
| 1 | IGY |
| 2 | IAY |
| 3 | IPVE |
| 4 | IEGTL |
| 5 | IVDI |
| 6 | VAPEEHPTL |
| 7 | IVY |
| 8 | IX¹Y, where X¹ is R, N, D, Q, E or K |
| 9 | IAX², where X² is Y, F or W |
| 10 | IGX³, where X³ is Y, F or W |
| 11 | LGY |
| 12 | LAY |
| 13 | LVY |
| 14 | LX¹Y, where X¹ is R, N, D, Q, E or K |
| 15 | LAX², where X² is Y, F or W |
| 16 | LGX³, where X³ is Y, F or W |

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety.

### REFERENCES

Chevrier, G., Mitchell, P. L., Rioux, L.-E., Hasan, F., Jin, T., Roblet, C. R., Marette, A. (2015). Low-Molecular-Weight Peptides from Salmon Protein Prevent Obesity-Linked Glucose Intolerance, Inflammation, and Dyslipidemia in LDLR-/-/ApoB100/100 Mice. The Journal of Nutrition, 145(7), 1415-22. http://doi.org/10.3945/jn.114.208215.
Roblet, C., Akhtar, M. J., Mikhaylin, S., Pilon, G., Gill, T., Marette, A., & Bazinet, L. (2016). Enhancement of glucose uptake in muscular cell by peptide fractions separated by electrodialysis with filtration membrane from salmon frame protein hydrolysate. Journal of Functional Foods, 22, 337-346. http://doi.org/10.1016/j.jff.2016.01.003.
Stumvoll, M., B. J. Goldstein, T. W. van Haeften, Type 2 diabetes: principles of pathogenesis and therapy., Lancet (London, England). 365 (2005) 1333-46. doi:10.1016/S0140-6736(05)61032-X.
Wild, S., G. Roglic, A. Green, R. Sicree, H. King, Estimates for the year 2000 and projections for 2030, Diabetes Care. 27 (2004) 1047-1053. doi: 10.2337/diacare.27.5.1047 Diabetes Care May 2004 vol. 27 no. 5 1047-1053.
Jin, T. (2012). Separation and purification of antidiabetic bioactive peptide from salmon and cod waste. Dalhousie University. Retrieved from https://dalspace.library.dal.ca/handle/10222/37804
Sievers, F., Wilm, A., Dineen, D., Gibson, T. J., Karplus, K., Li, W., Lopez, R., McWilliam, H., Remmert, M., Soding, J., Thompson, J. D., and Higgins, D. G. (2014). Fast, scalable generation of high-quality protein multiple sequence alignments using Clustal Omega. Molecular Systems Biology, 7(1), 539-539. https://doi.org/10.1038/msb.2011.75
Chen, Y. C., and Jaczynski, J. (2007). Protein recovery from rainbow trout (Oncorhynchus mykiss) processing byproducts via isoelectric solubilization/precipitation and its gelation properties as affected by functional additives. Journal of Agricultural and Food Chemistry, 55(22), 9079-9088. https://doi.org/10.1021/jf071992w
Crooks, G. E., Hon, G., Chandonia, J.-M., and Brenner, S. E. (2004). WebLogo: a sequence logo generator. Genome Research, 14(6), 1188-1190. https://doi.org/10.1101/gr.849004
Artimo, P., Jonnalagedda, M., Arnold, K., Baratin, D., Csardi, G., De Castro, E. Duvaud, S., Flegel, V., Fortier, A., Gasteiger, E., Grosdidier, A., Hernandez, C., loannidis, V., Kuznetsoy, D., Liechti, R., Moretti, S., Mostaguir, K., Redaschi, N., Rossier, G., Xenarios, I., and Stockinger, H. (2012). ExPASy: SIB bioinformatics resource portal. Nucleic Acids Research, 40(W1), W597-W603. https://doi.org/10.1093/nar/gks400
Strohalm, M., Hassman, M., Košata, B., and Kodíček, M. (2008). mMass data miner: an open source alternative for mass spectrometric data analysis. Rapid Communications in Mass Spectrometry, 22(6), 905-908. https://doi.org/10.1002/rcm.3444
Schneider and Stephens, 1990. Nucleic Acids Res. 1990 Oct. 25;18(20):6097-100. doi: 10.1093/nar/18.20.6097.
Li, Z., Paulson, A. T., and Gill, T. A., 2015. Encapsulation of bioactive salmon protein hydrolysates with chitosan-coated liposomes. J. Functional Foods 19 (Part A): 733-743.
Song, J., Wang, Q., Du, M., Li, T., Chen, B., and Mao, X. (2017). Casein glycomacropeptide-derived peptide IPPKKNQDKTE ameliorates high glucose-induced insulin resistance in HepG2 cells via activation of AMPK signaling. Molecular Nutrition & Food Research, 62(2).
Girgih, A. T., Udenigwe, C. C., Hasan, F. M., Gill, T. A., and Aluko, R. E., 2013. Antioxidant properties of Salmon (Salmo salar) protein hydrolysate and peptide fractions isolated by reverse-phase HPLC. Food Res Intl. 52: 315-322; and
Girgih, A. T., Nwachukwu, I. D., Hasan, F. M., Fagbemi, T. N., Malomo, S. A., Gill, T. A., and Aluko, R. E., 2016. Kinetics of in vitro enzyme inhibition and blood pressure-lowering effects of salmon (Salmo salar) protein hydrolysates in spontaneously hypertensive rats. J. Functional Foods 20: 43-53.

1. A peptide comprising:
(i) an amino acid sequence selected from:
(a) an amino acid sequence as shown in SEQ ID NO: 1 (IGY) or SEQ ID NO: 11 (LGY),
(b) an amino acid sequence as shown in SEQ ID NO: 7 (IVY) or SEQ ID NO: 13 (LVY),
(c) an amino acid sequence as shown in SEQ ID NO: 8 (IX1Y) or SEQ ID NO: 14 (LX1Y), where X¹ is a hydrophilic amino acid, optionally R, N, D, Q, E or K,
(d) an amino acid sequence as shown in SEQ ID NO: 9 (IAX2) or SEQ ID NO: 15 (LAX2), where X² is F or W, and
(e) an amino acid sequence as shown in SEQ ID NO: 10 (IGX3) or SEQ ID NO: 16 (LGX3), where X³ is Y, F or W; or
(ii) a peptide comprising at least 67% sequence identity with the amino acid sequence of any one of (a) to (e) that modulates glucose uptake,
wherein the peptide is less than 35 amino acids in length.

2. The peptide of claim 1, comprising the amino acid sequence shown in SEQ ID NO: 1.

3. The peptide of claim 1, consisting of the amino acid sequence shown in SEQ ID NO: 1.

4. The peptide of claim 1, wherein the peptide is less than 30, 25, 20, 15, 10, 8, 6, 5 or 4 amino acids in length, optionally wherein the peptide is modified for cell permeability, stability or bioavailability.

5. (canceled)

6. A composition comprising the peptide of claim 1 and a carrier.

7. A composition comprising the peptide of claim 1 and at least one additional peptide selected from:
(a) a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake,
(b) a peptide comprising (i) an amino acid sequence as shown in SEQ ID NO: 3 (IPVE); or (ii) a peptide comprising at least 75% sequence identity with the amino acid sequence as shown in SEQ ID NO: 3 that increases glucose uptake, and
(c) a peptide comprising (i) an amino acid sequence as shown in any one of SEQ ID NO: 4 (IEGTL), SEQ ID NO: 5 (IVDI) or SEQ ID NO: 6 (VAPEEHPTL); or (ii) a peptide comprising at least 67, 75, 80, or 90% sequence identity with the amino acid sequence as shown in any one of SEQ ID NOs: 4-6 that decreases hepatic glucose production,
and optionally a carrier.

8. A composition comprising: a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake and at least one additional peptide selected from:
(a) the peptide of claim 1,
(b) a peptide comprising (i) an amino acid sequence as shown in SEQ ID NO: 3 (IPVE); or (ii) a peptide comprising at least 75% sequence identity with the amino acid sequence as shown in SEQ ID NO: 3 that increases glucose uptake, and
(c) a peptide comprising (i) an amino acid sequence as shown in any one of SEQ ID NO: 4 (IEGTL), SEQ ID NO: 5 (IVDI) or SEQ ID NO: 6 (VAPEEHPTL); or (ii) a peptide comprising at least 67, 75, 80, or 90% sequence identity with the amino acid sequence as shown in any one of SEQ ID NOs: 4-6 that decreases hepatic glucose production,
and optionally a carrier.

9. An isolated fraction of salmon protein hydrolysate (SPH), wherein the fraction is obtained by fractionating salmon protein hydrolysate (SPH) by gel filtration chromatographic separation,
wherein the chromatographic separation is performed isocratically with 50 mM ammonium formate, pH 6.0 at a 0.1 mL/min flow rate, and
wherein the fraction modulates glucose uptake.

10. The fraction of claim 9, wherein the SPH is obtained by:
providing homogenized salmon;
precipitating protein from the homogenized salmon;
hydrolyzing the precipitated proteins to form a hydrolyzed solution; and
filtering the hydrolyzed solution using an ultrafiltration membrane to generate the SPH.

11. The fraction of claim 9, wherein the fraction has glucose uptake stimulating activity at 1 μg/mL and 1 ng/ml in cultured L6 myotubes.

12. The fraction of claim 9, wherein the fraction comprises at least one peptide of claims 1-4.

13. The fraction of claim 9, wherein the fraction comprises at least one peptide comprising or consisting of amino acid sequence shown in SEQ ID NO: 1 (IGY) or comprising or consisting of amino acid sequence shown in SEQ ID NO: 2 (IAY).

14. A composition comprising the fraction of claim 9 and optionally a carrier.

15. A method of increasing glucose uptake in a subject in need thereof, the method comprising administering to the subject:
the peptide of claim 1, or
a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

16. A method of regulating glucose levels, optionally regulating glucose within a normoglycemic range, in a subject in need thereof, the method comprising administering to the subject
the peptide of claim 1, or
a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

17. A method of treating diabetes, optionally type 1 or type 2 diabetes, in a subject in need thereof, the method comprising administering to the subject
the peptide of claim 1, or
a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

18. A method of treating metabolic syndrome (MS) by reducing hyperglycemia in a subject in need thereof, the method comprising administering to the subject
the peptide of claim 1, or
a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

19. A method of providing antioxidant treatment to a subject in need thereof, the method comprising administering to the subject
the peptide of claim 1, or
a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

20. A method of lowering blood pressure and/or treating or preventing hypertension in a subject in need thereof, the method comprising administering to the subject
the peptide of claim 1, or
a peptide comprising (i) an amino acid sequence as shown in SEQ ID NO: 2 (IAY) or SEQ ID NO: 12 (LAY); or (ii) a peptide comprising at least 67% sequence identity with the amino acid sequence as shown in SEQ ID NO: 2 or SEQ ID NO: 12 that modulates glucose uptake.

21. (canceled)

22. (canceled)

23. (canceled)

24. A method of obtaining the peptide of claim 1 and/or a peptide comprising (i) an amino acid sequence as shown in SEQ ID NO: 2 (IAY) or SEQ ID No: 12 (LAY) or (ii) a peptide comprising at least 67% sequence identity with the amino acid sequence as shown in SEQ ID NO: 2 or SEQ ID No: 12 that modulates glucose uptake, the method comprising:
providing a homogenized salmon frame or fraction;
precipitating proteins from the homogenized fraction;
hydrolyzing the precipitated proteins to form a hydrolyzed solution;
filtering the hydrolyzed solution using an ultrafiltration membrane to generate a filtrate; and
isolating the peptides from the filtrate, optionally isolating peptides of SEQ ID NO: 1, 11, 2 and/or 12 into separate fractions.
